(19) United States
(12) Patent Application Publication
(10) Pub. No.: US 2026/0345372 A1
(43) Pub. Date: Nov. 5, 2026

(54) MAXILLARY AIRWAY DEVICE

(71) Applicant: Selwyn GRUSD, Sandton (ZA)

(72) Inventor: Selwyn GRUSD, Sandton (ZA)

(21) Appl. No.: 19/102,711

(22) PCT Filed: Aug. 10, 2023
(86) PCT No.: PCT/IB2023/058086

§ 371 Date: Feb. 10, 2025

(30) Foreign Application Priority Data
Aug. 10, 2022 (ZA) 2022/08880
Oct. 14, 2022 (ZA) 2022/11277

(51) Int. Cl. A61F 5/56; B33Y 80/00

(52) U.S. Cl. CPC A61F 5/566; B33Y 80/00

(57) ABSTRACT

A maxillary airway device (10) typically used at least partially to clear an airway (68) of a user (16), to inhibit obstruction of the airway, particularly during sleep, thereby to treat sleeping disorders or conditions associated with restricted airflow. The device (10) includes a maxillary retaining arrangement (12) for releasably retaining the device (10) relative to a maxilla (14) of a person (16). The device (10) also includes at least a first formation (18) extending from or supported by the retaining arrangement (12) and comprising at least a first contact surface (20) configured for operatively contacting at least a first predetermined location (22) on a tongue (24) of the person (16). The first contact surface (20) urges or retains the tongue (24) of the person (16) towards or in a predetermined position.

### BACKGROUND TO THE INVENTION

This disclosure relates to ZA provisional patent application numbers 2022/08880 (“'880”) and 2022/11277 (“'277”) entitled “MAXILLARY AIRWAY DEVICE” to the same applicant hereof. The contents of '880 and '277 are incorporated herein in its entirety by reference.

This invention relates to a maxillary airway device. More particularly, the present invention relates to a maxillary airway device which is used at least partially to clear an airway of a user, to inhibit obstruction of the airway, particularly during sleep, thereby to treat sleeping disorders or conditions associated with restricted airflow, such as sleep apnoea and/or snoring. The device may also be used to improve airflow of persons with a deviated septum.

Sleep apnoea is a sleeping disorder in which normal breathing is intermittently or regularly interrupted, in other words, breathing frequently stops and starts. Sleep apnoea is the most common sleep-related breathing disorder, and some sources believe that up to 25% of males and 10% of females suffer from sleep apnoea.

Persons suffering from sleep apnoea may suffer from a range of symptoms, some of which relate directly to insufficient rest. Some symptoms include excessive daytime sleepiness, abrupt awakenings associated with gasping or chocking, morning headaches, difficulty in concentrating, mood changes, depression, irritability and the like.

Three types of sleep apnoea exist, namely obstructive sleep apnoea (the most prevalent type), central sleep apnea and complex sleep apnoea syndrome (combination of the first two).

Central sleep apnoea occurs when the brain does not sufficiently control muscles which are responsible for breathing, when asleep. Obstructive sleep apnoea occurs when the airway of a person is physically obstructed, which inhibits proper breathing. Obstructive sleep apnoea is therefore often associated with snoring, though snoring is not necessarily an indication that a person suffers from obstructive sleep apnoea. Obstructive sleep apnoea is caused by throat muscles, typically associated with a person's soft palate, uvula, tonsils and tongue, relaxing, causing the airway to narrow and close while breathing-in, resulting in a decrease of oxygen levels in the blood, and a build-up of carbon dioxide. A person's brain senses this impaired breathing and causes the person momentarily to wake up to correct its breathing. This cycle of momentarily waking up may be repeated between 5 and 30 times, or even more, per hour. Each cycle is short enough that the person will typically not be aware of it or remember it. The cycle is disruptive to the sleeping patterns and inhibits a person achieving restful sleep cycles, such as REM.

Some factors which are known to increase the likelihood of suffering from sleep apnoea, include excessive weight, a thicker neck circumference, narrowed airways, nasal obstructions, such as those caused by anatomical issues (such as a deviated septum), allergies, family history, use of tobacco, alcohol, sedatives or tranquilizers, and other medical conditions.

Current methods of treatment include the use of continuous positive airway pressure (CPAP) machines, which provides a predetermined flow of air and positive pressure to keep the airways open, mouth pieces which thrust the lower jaw forward during sleep, or even surgery. These methods of treatment achieve varying levels of success in preventing obstructive sleep apnoea and are not always compatible with all persons.

A need therefore exists for a device with which sleeping disorders or conditions associated with restricted airflow, such as sleep apnoea and/or snoring, or restricted airflow caused by a deviated septum, can be treated or at least alleviated. It is accordingly an object of the invention to provide a maxillary airway device that will, at least partially, address the above disadvantages or which will be a useful alternative to existing treatment devices of this kind.

### SUMMARY OF THE INVENTION

In accordance with a first aspect of the invention there is provided a maxillary airway device, comprising:
a maxillary retaining arrangement for releasably retaining the device relative to a maxilla of a person;
at least a first formation extending from or supported by the retaining arrangement and comprising at least a first contact surface configured for operatively contacting at least a first predetermined location on a tongue of the person, thereby urging or retaining the tongue of the person towards or in a predetermined position.

The predetermined location on the tongue may be located on an upper surface of a body of the tongue (corpus linguae), typically, between a tip of the tongue and a region containing vallate papillae, and towards a medial portion of the corpus linguae. The predetermined location may be proximate the vallate papillae. In some cases, the predetermined location may be vertically substantially in line with a junction between a hard palate and a soft palate of the person. In addition, or alternatively, the predetermined location may be vertically substantially in line with a lesser or greater palatine foramen of the person.

The predetermined location may be proximal to a line connecting second molars of the person.

In some examples, the predetermined location may be between 30 mm and 60 mm from a tip of the tongue. In practical terms, the predetermined location may be proximate a location where central incisors contact the tongue when the tongue is extended as far forwards out of the mouth, as possible and the incisors are used to bite down on the tongue. The predetermined location may be between about 50 mm and 60 mm from a tip of the tongue.

The predetermined position of the tongue is a neutral position of the tongue, a slightly forwards position, towards a bottom of the mouth, wherein the tip of the tongue contacts a lower incisor or a deflected position.

The first contact surface may operatively exert a downwards and/or forwards force on the tongue. The first contact surface may operatively deform the tongue or cause the tongue to be deformed from a neutral position of the tongue. Operative interaction between the tongue and the at least first contact surface may inhibit the tongue from lifting or moving towards a proximal or rear portion of the mouth, or towards a throat of the person.

The first formation may include a distal extension, which extends in a distal direction relative to the contact surface, and operatively, towards the tip of the tongue. The distal extension may have a slightly concave or cup-shaped surface which may cause suction to the tongue in use. The distal extension may operatively extend at least partially along a length of the tongue to exert a distributed force on the tongue, thereby retaining same in the predetermined position.

The first formation may include a stem portion. The distal extension may extend from the stem portion in cantilevered fashion. A brace formation may be located between the distal extension and the stem portion. A level of a tongue-facing surface of the distal extension may be spaced from a level of the contact surface by about 15 mm. A tip or end of the distal extension may extend about 30 mm distally from the contact surface. The contact surface and/or distal extension may have a width of about 10 mm.

A level of the contact surface may, in use, be spaced from a palate of the person by between 50 mm and 60 mm. The contact surface may, in use, be spaced about 40 mm from maxillary incisors of the person. A connecting line between the contact surface and incisors of the person may be between 50 mm and 60 mm.

The retaining arrangement may be configured to retain the device releasably relative to maxillary teeth of the person, an alveolar process and/or a maxillary dental arch (maxillary gums or gingiva) of the person. The retaining arrangement may comprise first and second retaining portions in the form of first and second retaining trays being substantially U-shaped in cross-section. The first and second retaining portions may be discrete portions. Alternatively, the first and second portions may be integrally formed to form a substantially U-shaped tray viewed from a bottom.

The retaining arrangement may either be configured to retain the device releasably relative to first and second sets of maxillary teeth respectively, the first and second sets of maxillary teeth located on opposite sides of the mouth of the person or may be configured to retain the device releasably relative to first and second portions of a maxillary gingiva or gum of the person, the first and second portions of gingiva located on opposite sides of the mouth of the person.

The first and second sets of maxillary teeth may comprise some of first and second sets of maxillary molars and premolars, maxillary canine teeth, and/or maxillary third molars.

The first and second retaining portions may extend beyond the second molars and/or the third molars at least partially over maxillary posterior tuberosities.

The device may be integrally formed from a medical grade and slightly elastic polymeric material. The material may be a soft or substantially elastic material, a dual laminate material, printable resin or dental wire. Furthermore, the material may be acrylic (polymethyl methacrylate), nylon, natural or synthetic rubber, silicon or a silicon-based material.

The device may further comprise a cross-member, bridge or brace, connecting opposite sides of the retaining arrangement or first and second retaining portions thereof. The stem portion of the formation may extend or project from the cross-member, bridge or brace. The cross-member, bridge or brace may comprise two or four arms.

Further according to the first aspect of the invention, the device may comprise a second formation which may extend from or be supported by the retaining arrangement. The second formation may comprise a second contact surface configured for operatively contacting a second predetermined location on a tongue of the person and wherein operative interaction between the first and second contact surfaces and the first and second predetermined locations respectively, urges or retains the tongue of the person towards or in the predetermined position.

The first and second formations may be spaced apart to define a spacing distance therebetween. The first and second formations may be formed by or supported by first and second cross-members, bridges or bases respectively, which extend between opposite sides of the retaining arrangement or first and second retaining portions thereof. The first and second cross-members, bridges or bases may be spaced apart and may define a spacing distance therebetween. The second predetermined location may be a location on an underside of the tongue and wherein the first and second contact surfaces operatively contact opposite sides of the tongue, such that at least a portion of the tongue is operatively received within the spacing distance.

The first formation may comprise a first set made up of more than one protrusion. The first contact surface may be defined across the protrusions of the first set. The first formation may include an operatively throat-facing or rearward extending grip or hook member, defining a gripping or hooking contact surface, which may be shaped and configured to contact a third predetermined location on the tongue. The third location may be towards a rear portion of the tongue or towards a throat of the person.

A palate-facing surface of the gripping or hook member may include a bifurcation. The bifurcation may extend non-linearly and at least in a portion thereof, may be spaced from an operative sagittal plane.

The device may include one or more portions with surface finishes such as bulbosities, bulges, ridges, bifurcations, hollows, or grooves.

In accordance with a second aspect of the invention there is provided a method of manufacturing a device according to claim 1, the method comprising the steps of:
S1) determining a shape, profile, size and/or configuration of a structure associated with a maxilla of a person;
S2) determining a predetermined location on a tongue of the person;
S3) constructing the device, by:
   S3.1) constructing a retaining arrangement based on the determined shape, profile, size and/or configuration of the structure; and
   S3.2) constructing a formation based on the predetermined location.

Step S1) may comprises taking an intraoral scan of a mouth of the person or taking a physical or dental impression of the mouth of the person. Step S3 may be conducted using an additive manufacturing process. Alternatively, step S3 may comprise creating a mould of a portion of a mouth of the person and forming the retaining arrangement and formation on the mould.

### BRIEF DESCRIPTION OF THE DRAWINGS

The invention will now be described in more detail, by way of example only, with reference to the accompanying drawings in which:

FIG. 1 shows a perspective view of a maxillary airway device in accordance with the invention;

FIG. 2 shows a perspective view of the maxillary airway device of FIG. 1, in use, fixed relative to a maxilla of a person, which is partially shown;

FIG. 3 shows a bottom view of the maxillary airway device of FIG. 1, again shown in use, fixed relative to a maxilla of a person, which is partially shown;

FIG. 4 shows a side view of the maxillary airway device of FIG. 1;

FIG. 5 shows a side view of a person, with a mouth and throat portion of the person shown in section to reveal details of the mouth, and particularly, the airway, of the person, when in a relaxed state, and without the use of the maxillary airway device of FIG. 1;

FIG. 6 shows a side view of maxillary airway device of FIG. 1, in use, positioned in the mouth of a person as shown in FIG. 5;

FIG. 7 shows a detailed view of the maxillary airway device of FIG. 1, in use, positioned in the mouth of the person, as shown in FIG. 6;

FIG. 8 shows a perspective view of a maxillary airway device in accordance with an alternative embodiment of the invention;

FIG. 9 shows a perspective view of the maxillary airway device of FIG. 8, in use, fixed relative to a maxilla of a person, which is partially shown;

FIG. 10 shows a bottom view of the maxillary airway device of FIG. 8, again shown in use, fixed relative to a maxilla of a person, which is partially shown;

FIG. 11 shows a side view of the maxillary airway device of FIG. 8;

FIG. 12 shows an enlarged side view of the maxillary airway device of FIG. 8, in use, in a mouth of a person, in which dimensions of the device is indicated;

FIG. 13 shows a perspective view of a maxillary airway device in accordance with a further embodiment of the invention;

FIG. 14 shows a perspective view of the maxillary airway device of FIG. 13, in use, fixed relative to a maxilla of a person, which is partially shown;

FIG. 15 shows a bottom view of the maxillary airway device of FIG. 13, again shown in use, fixed relative to a maxilla of a person, which is partially shown;

FIG. 16 shows a side view of the maxillary airway device of FIG. 13;

FIG. 17 shows a side view of a person, with a mouth and throat portion of the person shown in section to reveal details of the mouth, and particularly, the airway, of the person, wherein the maxillary airway device of FIG. 13 is positioned in the mouth of the person;

FIG. 18 shows a perspective view of a maxillary airway device in accordance with another further embodiment of the invention;

FIG. 19 shows a perspective view of the maxillary airway device of FIG. 18, in use, fixed relative to a maxilla of a person which is partially shown;

FIG. 20 shows a perspective view of a maxillary airway device in accordance with yet another further embodiment of the invention;

FIG. 21 shows a perspective view of the maxillary airway device of FIG. 20, in use, fixed relative to a maxilla of a person which is partially shown;

FIG. 22 shows a bottom view of the maxillary airway device of FIG. 18 or 20, again shown in use, fixed relative to a maxilla of a person, which is partially shown;

FIG. 23 shows a side view of the maxillary airway device of FIG. 18 or 20;

FIG. 24 shows a side view of a person, with a mouth and throat portion of the person shown in section to reveal details of the mouth, and particularly, the airway, of the person, wherein the maxillary airway device of FIG. 18 or 20 is positioned in the mouth of the person;

FIG. 25 shows a perspective view of a maxillary airway device in accordance with yet a further embodiment of the invention;

FIG. 26 shows a perspective view of the maxillary airway device of FIG. 25, in use, fixed relative to a maxilla of a person, which is partially shown;

FIG. 27 shows a bottom view of the maxillary airway device of FIG. 25, again shown in use, fixed relative to a maxilla of a person, which is partially shown;

FIG. 28 shows a side view of the maxillary airway device of FIG. 25;

FIG. 29 shows a top view of the maxillary airway device of FIG. 25, together with a detailed perspective view of a portion thereof;

FIG. 30 shows a side view of a person, with a mouth and throat portion of the person shown in section to reveal details of the mouth, and particularly, the airway, of the person, wherein the maxillary airway device of FIG. 25 is positioned in the mouth of the person;

FIG. 31 shows a perspective view of a maxillary airway device in accordance with yet a further embodiment of the invention;

FIG. 32 shows a perspective view of the maxillary airway device of FIG. 31, in use, fixed relative to a maxilla of a person, which is partially shown;

FIG. 33 shows a bottom view of the maxillary airway device of FIG. 31, again shown in use, fixed relative to a maxilla of a person, which is partially shown;

FIG. 34 shows a side view of the maxillary airway device of FIG. 31;

FIG. 35 shows a top view of the maxillary airway device of FIG. 31, together with a detailed perspective view of a portion thereof;

FIG. 36 shows a side view of a person, with a mouth and throat portion of the person shown in section to reveal details of the mouth, and particularly, the airway, of the person, wherein the maxillary airway device of FIG. 31 is positioned in the mouth of the person;

FIG. 37 shows a perspective view of the maxillary airway device of FIG. 31, in which certain surface finishes and features are indicated;

FIG. 38 shows a perspective view of the maxillary airway device of FIG. 37, in use, fixed relative to a maxilla of a person, which is partially shown;

FIG. 39 shows a bottom view of the maxillary airway device of FIG. 37, again shown in use, fixed relative to a maxilla of a person, which is partially shown;

FIG. 40 shows a side view of the maxillary airway device of FIG. 37;

FIG. 41 shows a top view of the maxillary airway device of FIG. 37, together with a detailed perspective view of a portion thereof;

### DESCRIPTION OF THE ILLUSTRATED EMBODIMENTS

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being conducted in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Unless specified or limited otherwise, the terms “mounted”, “connected”, “engaged” and variations thereof are used broadly and encompass both direct and indirect mountings, connections, supports, and couplings and are thus intended to include direct connections between two members without any other members interposed therebetween and indirect connections between members in which one or more other members are interposed therebetween. Further, “connected” and “engaged” are not restricted to physical or mechanical connections or couplings. Additionally, the words “lower”, “upper”, “upward”, “down” and “downward” designate directions in the drawings to which reference is made. The terminology includes the words specifically mentioned above, derivatives thereof, and words or similar import. It is noted that, as used in this specification and the appended claims, the singular forms “a,” “an,” and “the,” and any singular use of any word, include plural referents unless expressly and unequivocally limited to one referent. As used herein, the term “include” and its grammatical variants are intended to be non-limiting, such that recitation of items in a list is not to the exclusion of other like items that can be substituted or added to the listed items.

Referring to the drawings, in which like numerals indicate like features, non-limiting examples of a maxillary airway device, or simply “device” for the sake of brevity, in accordance with the invention, is generally indicated by reference numeral 10. The device 10 comprises a retaining arrangement 12, with which the device 10 is releasably fixed, retained or attached relative to a maxilla 14 (upper fixed jawbone) of a person 16.

As will become apparent from what follows, the retaining arrangement 12 may take various forms depending on the anatomical structures of the person 16, and for example, whether the device 10 is fixed to teeth, gums or the like of the person 16. However, generally, the device 10 is (releasably) fixed, retained or attached relative to the maxilla 14, meaning that displacement of the device 10 relative to the maxilla 14 is limited or inhibited, if not avoided altogether. The maxilla 14 can therefore be viewed as a stationary base or foundation to or relative to which the device 10 is releasably fixed—directly or indirectly.

The device also comprises a formation, which in the examples shown herein takes the form of a protrusion 18 which extends, directly or indirectly, from the retaining arrangement 12, in downward fashion (operatively, and therefore relative to the mouth of the person). Details of how the protrusion 18 is fixed or attached relative to the retaining arrangement, are provided below.

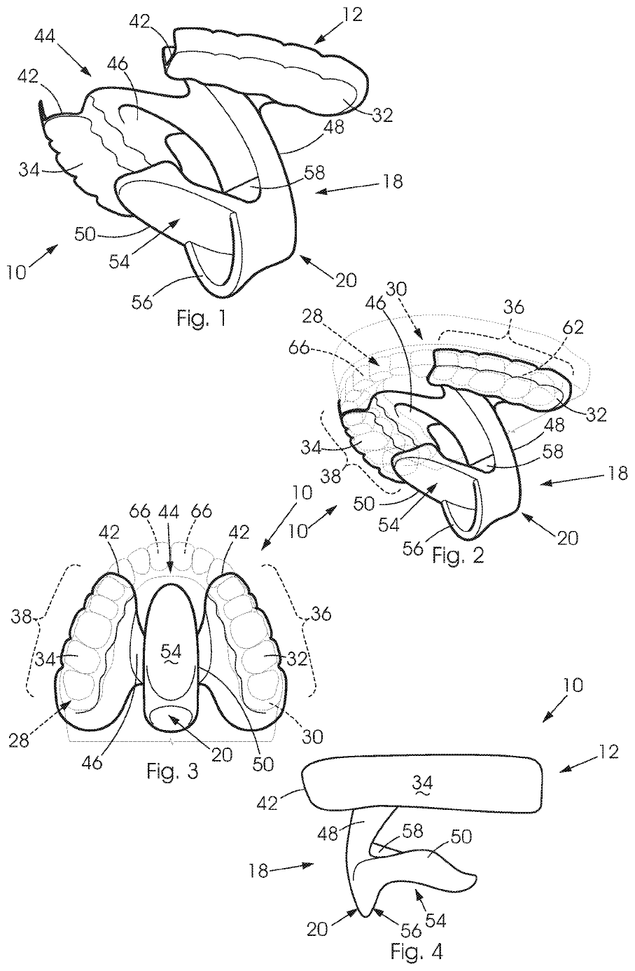

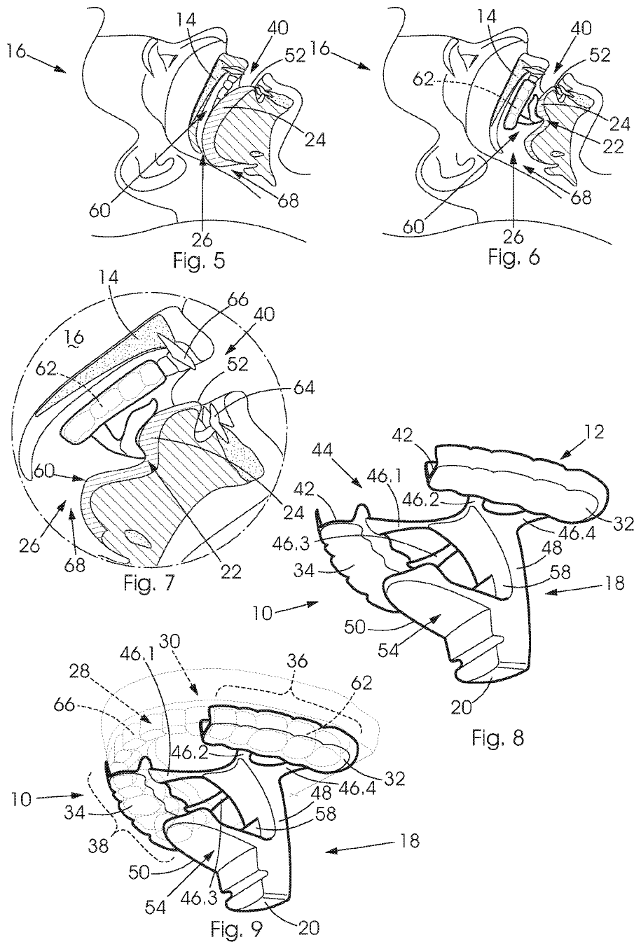

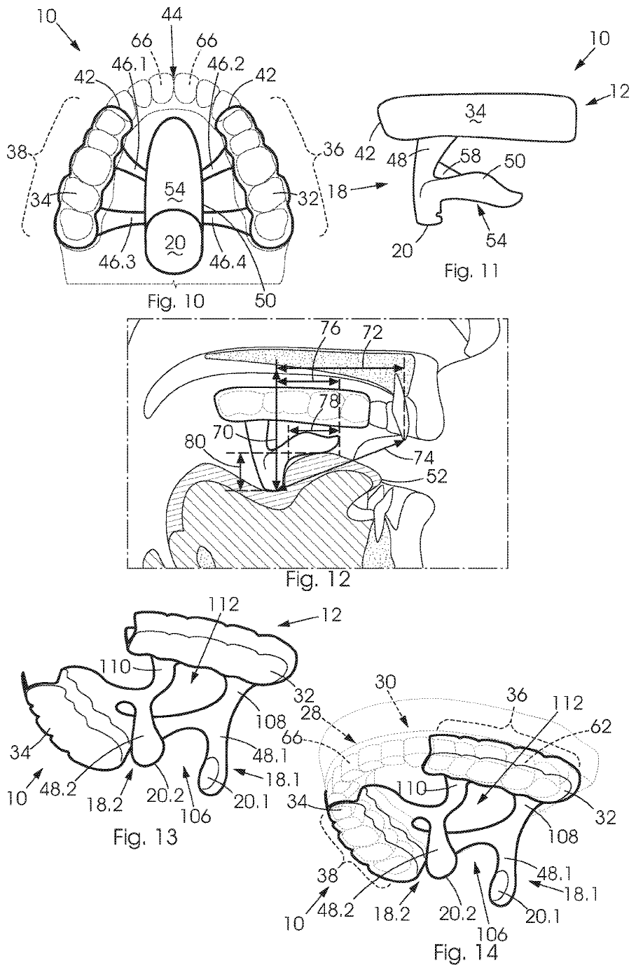

The protrusion 18 comprises a contact surface 20, which is shaped, positioned and configured, when used by the person 16 as discussed more fully below to contact a predetermined location 22 on a tongue 24 of the person 16. FIGS. 1 to 7 show a first embodiment of the invention, where the contact surface 20 takes the form of an edge portion of the protrusion 18. FIGS. 8 to 12 show a second embodiment of the invention, in which the contact surface 20 comprises an upper surface of the protrusion 18. Other forms are discussed below.

Throughout this disclosure, dimensions or relative positions disclosed in respect of the contact surface 20, will be taken to refer to or be taken from a specific point, such as a central point, located on the contact surface 20. As discussed more fully below, the predetermined location 22, and the way in which the contact surface 20 makes contact therewith, are very specific. The interaction or contact between the contact surface 20 and the predetermined location 22 on the tongue 24 urges the tongue 24 of the person 16 towards a predetermined location (as is typically shown in FIGS. 6 and 7), retains or keeps the tongue 24 in this predetermined location and/or inhibits the tongue 24 from being displaced away from this predetermined location, or towards an undesirable position or location, which typically, is towards a throat 26 of the person 16. This is elaborated on below. It will be understood that the contact surface 20 may, in use, urge the predetermined location 22 away from a position in which it is normally located, absent use of the device 10. The tongue may thus be inhibited from moving from the predetermined position.

The retaining arrangement 12 may take various forms without departing from the spirit and scope of the present disclosure. Therefore, and as discussed more fully below, the retaining arrangement is provided for releasably fixing the device 10 to maxillary teeth 28 or, in cases where the person does not possess maxillary teeth, to an alveolar process or maxillary dental arch 30 (maxillary or dental gingiva or gum) of the person 16. The alveolar process will be referred to as the maxillary gum 30 for the sake of brevity herein.

### Fixing to the Teeth

The example embodiment of the retaining arrangement 12 shown in the figures, where the retaining arrangement 12 fixes or attaches the device 10 directly to maxillary teeth 28 of the person 16, is now discussed in more detail. The retaining arrangement 12 comprises a first and second retaining portion (32, 34), which are configured releasably to retain the device 10 relative to a first and second set of maxillary teeth 28 (the first and second sets of teeth 28 are indicated by reference numerals 36 and 38 respectively). The first and second sets (36, 38) are situated on opposite sides of the mouth 40 of the person 16 and comprise of first and second sets of maxillary molars and premolars but may also include third molars (when present) and maxillary canine teeth. Furthermore, the first and second retaining portions may extend beyond the molars and may contact maxillary posteriors tuberosities. Therefore, each of the first and second retaining portions (32, 34) may extend, at least partially, over a maxillary canine, first premolar, second premolar, first molar, second molar, third molar (if present) and posterior tuberosities.

The first and second retaining portions (32, 34) are substantially similar in nature (although substantially mirror images of each other) and features discussed below in respect of the first retaining portion 32 will be taken to apply mutatis mutandis to the second retaining portion 34, unless otherwise stated. The first retaining portion 32 takes the form of a fixing or retaining tray within which the teeth 28 of the first set 36 are received. In section, the retaining tray is substantially shaped in the form of an inverted U-shape. The retaining tray is shaped commensurate a profile of the set 36, such that the tray fits over the teeth 28 snugly with a fine tolerance, causing same to grip onto the teeth, or to suck onto the teeth. The retaining tray is therefore moulded or shaped based on the profile of the set 36.

The manufacturing process of the tray may include creating a mould of the maxillary teeth 28 of the person 16, or alternatively, performing an intra-oral scan of the mouth 40 (and particularly of the maxillary and mandibular arches) of the person 16. When using an intra-oral scan, the results can be used as an input to computer software which may be configured to design/specify the physical dimensions, shape and configuration of the device, based on the scan and the parameters detailed herein. The device may then be manufactured by way of additive manufacturing (using the output generated by the software). This process may be relative quick and may take place while the patient waits. Alternatively, the device may be manufactured by hand, in which case required dimensions and configurations of the device may be provided to the manufacturer by the software or may be determined from the mould (if relevant).

The first and second retaining portions (32, 34) are discrete portions, in the sense that anterior ends 42 of the portions (32, 34) do not meet up and therefore define an opening 44 therebetween. Therefore, in the embodiment shown, maxillary incisors of the person 16 are not covered by the retaining arrangement 12.

In some example embodiments, such as shown in FIGS. 20 and 21, upper parts of the first and second retaining portions (32, 34) are hollowed or open-ended to define openings 100, to reduce an overall thickness of the retaining portions (32, 34). This may be applied to any of the embodiments shown or discussed herein.

### Fixing to the Maxillary Gum

As mentioned, though not shown, an embodiment where the retaining arrangement 12 is configured for fixing or attaching the device directly to the maxillary gum 30, and therefore, in cases where the person 16 does not have teeth 28 (edentulous persons), is possible, and falls within the scope of the present disclosure.

In such a case the retaining arrangement 12 still comprises first and second retaining portions. These differ from the first and second retaining portions (32, 34) discussed above in that the retaining trays are shaped commensurate opposing portions of the maxillary gum 30 or the maxillary fitting surface of a denture. These opposing portions of the maxillary gum 30 may be portions located proximate positions where molars and premolars would have been located, should the person 16 have had teeth 28. A material from which the first and second retaining portions is manufactured may be softer and more pliable, to prevent irritation or damage to the maxillary gum 30, and to improve suction between the retaining portions and the maxillary gum 30. Also, in some cases the use of an adhesive substance, such as a gel, may be required to retain the retaining portions in position relative to the maxillary gum 30.

If required, the device 10 can replace or replicate a whole denture of an edentulous person. In such cases, the device will be designed to be incorporated into a copy or replica of the persons denture or into a new maxillary, palatal denture base.

In other respects, such as the retaining portions having inverted U-shapes in section and being discrete portions the retaining portions of this embodiment of the retaining arrangement 12 may be substantially similar to the retaining portions (32, 34) discussed above in respect of the embodiment shown in the figures.

In a variation of either embodiment discussed above, the first and second retaining portions may be integrally formed, such that the retaining arrangement 12 may comprise a single, substantially U-shaped formation (when viewed from the bottom), which covers maxillary incisors and molars of the person 16. This is not shown.

The device 10 includes a cross-member, bridge or brace 46, which extends between and connects the first and second retaining portions (32, 34). The specific shape and configuration of the cross-member or bridge 46 is discussed more fully below.

A stem portion 48 of the protrusion 18 extends from the cross-member 46, in downward fashion (operatively). The cross-member 46 therefore serves as a base portion of the device 10 and supports the stem portion 48.

In some embodiments, the protrusion 18 includes a distal extension 50, which extends distally from the stem portion 48. In use, the distal extension 50 extends in a direction towards the tip 52 of the tongue 24.

An operatively tongue-facing surface 54 of the distal extension 50 is slightly hollow, concave or cup-shaped. It may also include a dedicated cup-shaped formation 56. The concave shape of the surface 54 and/or the cup-shaped formation 56 are provided for causing suction between itself and the surface of the tongue 24, in use, to aid in fixing the tongue relative to the device 10. The suction is therefore distributed over a relatively large contact area of the surface 54, which ensures proper fixation to the tongue 24, and also lessens discomfort or fatigue which may be associated with the interaction between the device 10 and the tongue. Also, to this end, edges of the surface 54 are rounded.

The distal extension 50 extends from the stem portion 48 in cantilevered fashion. A brace formation 58 extends between the distal extension 50 and the stem portion 48, to support the distal extension 50 and lessen stress caused by a moment about the connection point between the distal extension 50 and the stem portion 48. The distal extension 50 is spaced from the contact surface 20 which typically, is located towards an end of the stem portion 48. A level of the surface 54 may typically be spaced (indicated by a sixth dimension 80 in FIG. 12) by about 15 mm from the contact surface 20. It will be appreciated that the contact surface 20 may, in use, displace or force the predetermined location 22 downward by about 15 mm relative to a normal location of an upper surface of the tongue 24 (which is associated with and typically provided in contact with the surface 54). This may serve to “lock” or hold the tongue in position. A ridge or groove (not shown) may be located between the contact surface 20 and the lateral extension 50 to improve grip to the tongue.

The device 10 is integrally formed from a slightly elastic polymeric material. Typically, the material may comprise soft or substantially elastic material, a dual laminate material or printable resins. The material may comprise acrylic (polymethyl methacrylate), nylon, natural or synthetic rubber, silicon, or a silicon-based material.

As mentioned, the predetermined location 22 is not arbitrary, and is specifically selected based on an anatomy of a specific person 16. That said, it has been found that the dimensions provided by way of example herein, are compatible with a large complement of adults (both male and female). Also, provision is made for deviating slightly from the exact dimensions provided herein, should the anatomy of the person 16 so dictate. Again, the specific dimensions may be confirmed by way of an intra-oral scan of the mouth 40 or physical cast taken from mouth 40 of the person 16.

It will furthermore be appreciated that the specific configuration, dimensions, use and functioning of the device 10 are all affected by combined effects of the specific location 22, and dimensions and configurations of the protrusion 18 and contact surface 20.

The predetermined location 22 is located on an upper surface of the body of the tongue 24. The predetermined location lies between the tip 52 of the tongue 24 and an area 60 of the tongue towards a rear of the mouth 40, or towards the throat 26, and more particularly, an area where vallate papillae is located. The predetermined location 22 may be slightly in front of the area 60 and therefore the vallate papillae.

The predetermined location 22 is typically aligned with a line running through a junction between a hard palate and soft palate (not shown) of the person 16. Additionally, or alternatively, the predetermined location 22 may be substantially vertically aligned with lesser or greater palatine foramen (again not shown) of the person 16.

Yet further alternatively, or in addition, the predetermined location may be substantially aligned with a plane running through second molars 62 of the person 16 or a location where second molars would typically be located (in cases where the person 16 does not have teeth).

It will be appreciated that the above detail pertaining to the predetermined location 22 is determined when the tongue 24 is provided in a normal relaxed position, which is a position in which the tongue 24 lies substantially flat in the mouth. The tip 52 of the tongue 24 is therefore, typically, pressed-up against the lower or mandibular teeth 64 of the person 16.

In some cases, the predetermined location 22 may be around 53 mm measured from the tip 52 of the tongue 24. It is believed that this dimension may, potentially, vary in a range of 30 mm to 60 mm, or even a range of 35 mm to 55 mm, depending on anatomy of the person 16 or the configuration of the device 10.

Alternatively, or in addition, the predetermined location 22 may be a location where central incisors 66 contact the upper surface of the tongue 24, when the tongue 24 is stuck out of the mouth 40 as far as possible, and the mouth 40 is closed onto the tongue 24, so as to bite down on the tongue 24 using the incisors 66.

By contacting the predetermined location 22 with the contact surface, the tongue 24 can be held in, or urged towards, the predetermined position. The predetermined position in which the tongue is held or towards which the tongue is urged, is furthermore influenced by a distance by which the contact surface 20, and also the distal extension 50, are spaced from the retaining arrangement 12 (therefore, directly or indirectly determined by a length of the stem portion 48).

A number of important dimensions of the device according to the first embodiment are shown in FIG. 12. Again, it is stressed that variations to these dimensions may occur, based on the specific anatomy of the person 16. All dimensions mentioned herein will be taken to allow for a tolerance or small variation of between plus or minus 4 mm, unless otherwise stated or the context dictates otherwise.

A first dimension 70, being a spacing between the contact surface 20 and the palate, or more particularly, the lesser or greater palatine foramen, may be about 53 mm. A second dimension 72, being a distal spacing between the contact surface 20 and the maxillary central incisors 66, may be about 40 mm. A third dimension 74, associated with a connecting line between the contact surface 20 and the maxillary central incisors 66 may be about 53 mm. A fourth dimension 76, being a distal extent of the distal extension 50 (in other words, distal spacing between the contact surface 20 and a tip of the distal extension 50) may be about 30 mm. A fifth dimension 78, relating to a length of the distal extension 50 (measured between a base and the tip of the distal extension 50) may be about 20 mm.

In use, the person 16 inserts the device 10 into the mouth 40 and secures it in position relative to the maxilla 14, as discussed above. The mouth 40 is opened wide during this procedure, and the tongue 24 is intentionally held in a downward and forwards position. Once the device 10 is secured in position, the mouth 40 is closed, and the contact surface 20 and surface 54 of the distal extension 50 are brought into contact with the tongue 24. The contact surface 20 is therefore now provided in contact with the predetermined location 22. The tongue 24 is now held in the predetermined position and inhibited from moving away from the predetermined position. When the tongue is in the predetermined position, the airway 68 of the person 16 is substantially unobstructed.

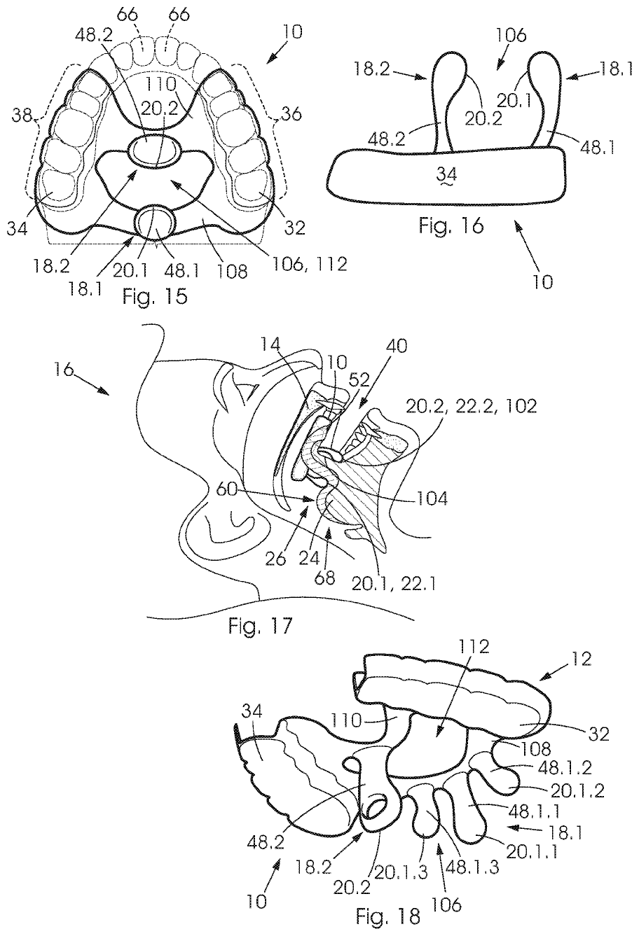

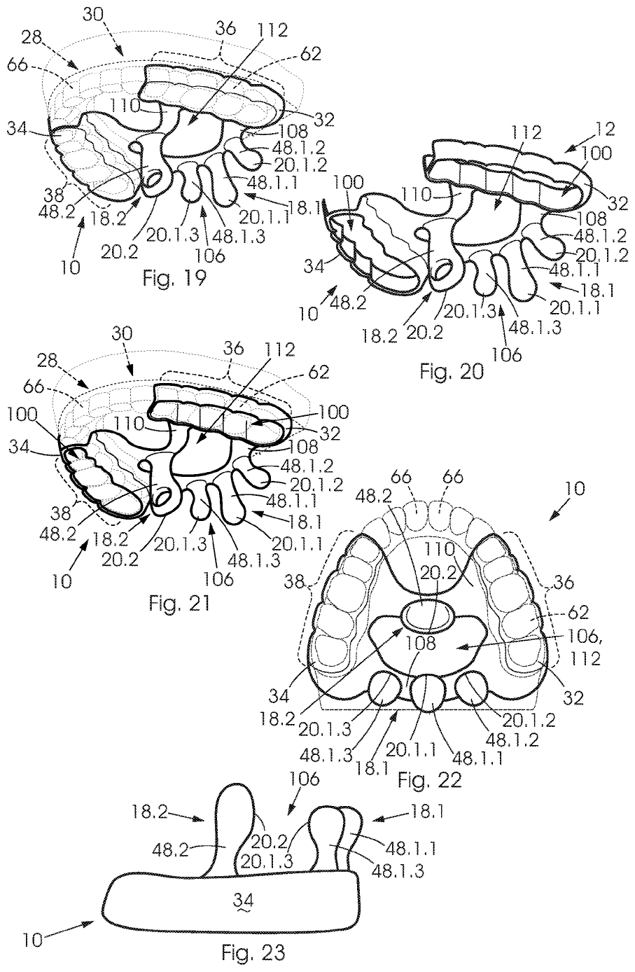

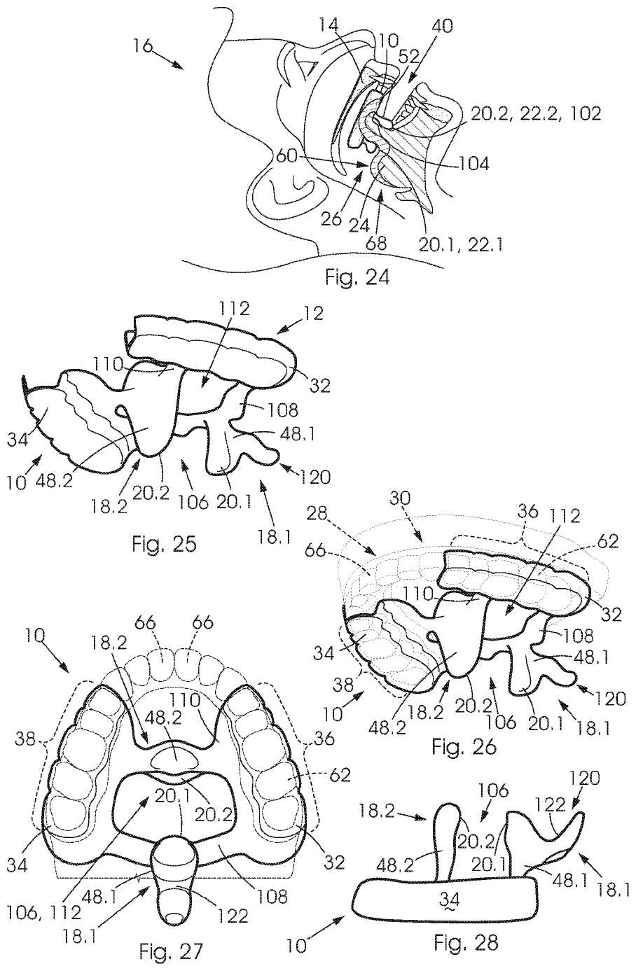

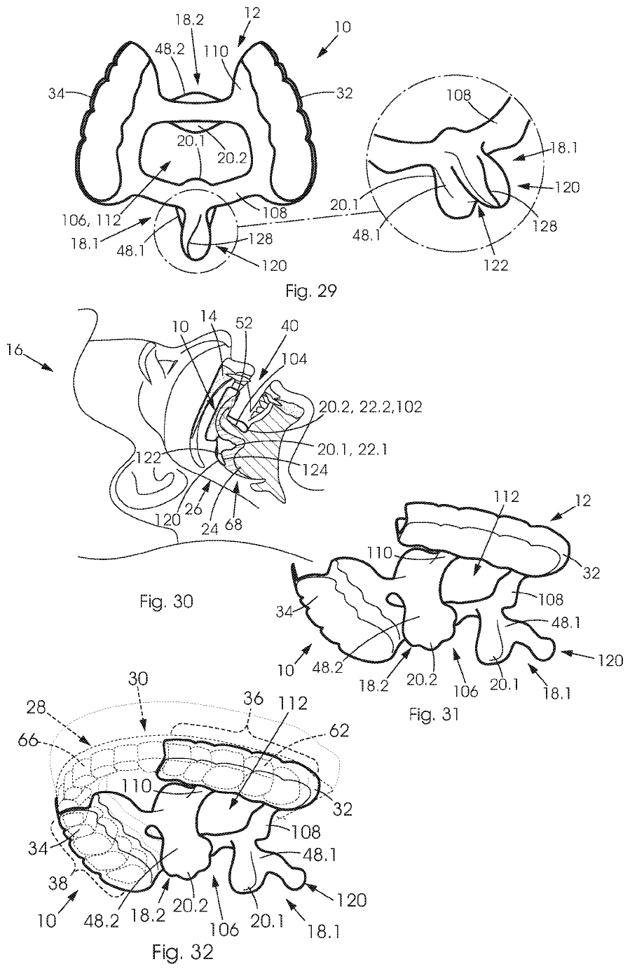

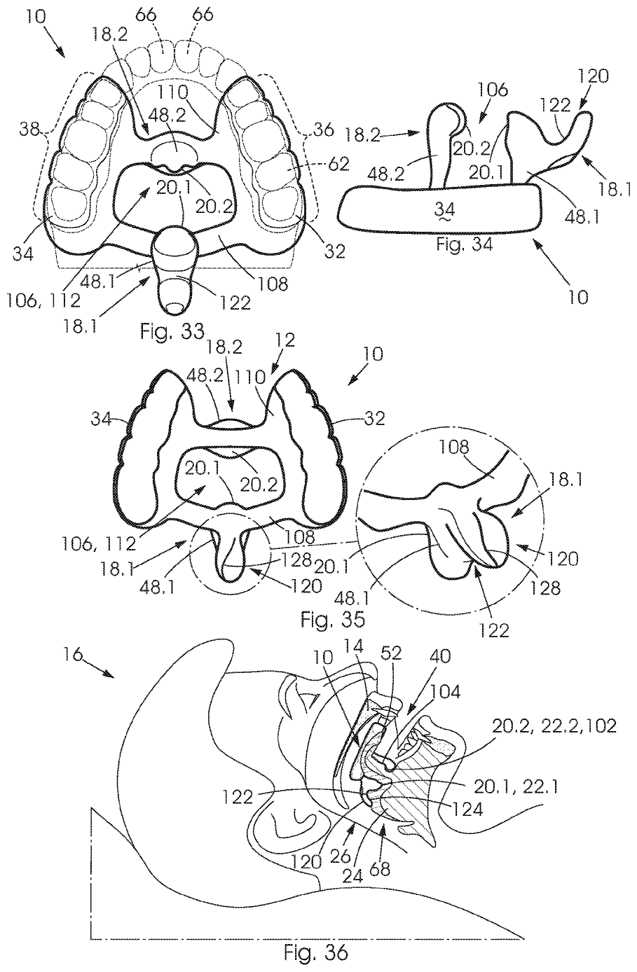

Further alternative embodiments of the invention are shown in FIGS. 13 to 41. Like numerals indicate like features where relevant. Features as described above with references to the embodiments of FIGS. 1 to 12 may be present and may be and may function substantially similarly in the embodiments of FIGS. 13 to 41, unless stated otherwise or the context dictates otherwise. The below description will focus only on main differences between the various embodiments.

In the alternative embodiments, the protrusion 18 takes the form of a first protrusion 18.1 and the device 10 furthermore comprises a second protrusion 18.2. The contact surface 20 as described before therefore takes the form of a first contact surface 20.1 which is associated with the first protrusion 18.1. The second protrusion 18.2 is associated with a second contact surface 20.2. The first and second protrusions (18.1, 18.2) are formed by first and second stem portions (48.1, 48.2).

The first contact surface 20.1 is still configured to contact the predetermined location 22 on the tongue 24 (now taking the form of a first predetermined location). The first predetermined location 22.1 is substantially similar to the predetermined location 22 described above and the dimensions and/or method of determining said predetermined location will not be repeated here. Similarly, the second contact surface 20.2 is configured to contact a second predetermined location 102 on the tongue 24. Typically, the second predetermined location 102 is situated on a bottom surface 104 of the tongue and is substantially opposite to the first predetermined location 22.1.

The first and second protrusions (18.1, 18.2) and are spaced apart and the first and second contact surfaces (20.1, 20.2) face each other and define a spacing distance 106 therebetween. Typically, the first and second protrusions (18.1, 18.2) diverge slightly along their lengths, such that the spacing distance 106 increases along the length of the first and second protrusions (18.1, 18.2). This facilitates insertion of the tongue between the first and second contact surfaces (20.1, 20.2), whilst exerting a slight “gripping” force towards the tip of the tongue, thereby to facilitate retaining the tongue in position.

The first and second protrusions (18.1, 18.2) extend from or are supported by first and second cross-members, bridges or bases (108, 110), which extend between the first and second retaining portions (32, 34). Again, first and second cross-members, bridges or bases (108, 110) are spaced apart and define spacing distance 112 therebetween.

It follows that the tongue 24 (or at least a portion thereof) is located within the spacing distances (106, 112) and therefore extend between the first and second contact surfaces (20.1, 20.2). The tongue 24 is therefore “gripped” or retained between the first and second contact surfaces (20.1, 20.2).

In some cases, the predetermined position towards or in which the tongue 24 is urged or retained is therefore a position in which the tip 52 of the tongue faces upwards towards the palate. The tongue 24 therefore deflects or extends around the first protrusion 18.1. Alternatively, as shown in the figures, the tip 52 of the tongue 24 may point substantially forwards, and the tongue may therefore deflect or extend around the first protrusion 18.1, and over the second cross member, bridge or base 110.

The device 10 is installed by tilting the device 10 forwards, such that the operative bottom end of the device 10 faces towards the mouth 40 of the person 16. The person 16 sticks out its tongue 24 and advances the device 10 over the tip 52 of the tongue 24 such that the tip 52 extends thorough the spacings (106 and 112). The first and second contact surfaces (20.1, 20.2) now contact the first and second predetermined locations (22.1, 22.2) on the upper and lower surfaces of the tongue 24 respectively and grips the tongue 24 therebetween. Next, the device 10 is tilted backwards (towards the operative orientation) and installed by placing the first and second retaining portions (32, 34) in position as hereinbefore described. This action therefore deflects the tongue 24 into the predetermined position described. Spatial constraints within the mouth and the gripping force exerted by the first and second contact surfaces (20.1, 20.2) inhibit the tongue from moving out of the predetermined position. This also inhibits the tongue from moving backwards towards the throat 26 and therefore inhibits interference with or blocking of the airway 68.

The embodiment of FIGS. 18 to 24 show a slight variation to this embodiment. Here, the first protrusion 18.1 comprises a first set, comprising three stems (marked 48.1.1 to 48.1.3). Each of these three stems comprises a contact surface (marked 20.1.1 to 20.1.3) respectively. The three contact surfaces (20.1.1, 20.1.2, 20.1.3) collectively make up the first contact surface 20.1 and are provided for increasing and distributing the contact surface and interaction between the first contact surface 20.1 and the tongue 24.

The embodiment of FIGS. 25 to 30 show a further alternative embodiment, wherein the first protrusion 18.1 additionally includes an operatively throat-facing or rearward extending grip or hook member 120. The grip or hook member 120 defines a gripping or hooking contact surface 122, which is shaped and configured, operatively, to contact a third predetermined location 124 of the tongue 24. This third predetermined location 124 is situated towards a rear portion of the tongue, towards the throat 26. It has been found that this grip or hook member 120 may provide a particularly effective means of inhibiting the tongue 24, and particularly the rear portion of the tongue, from blocking or occluding the throat 26. A surface (not indicated) of the grip or hook member 120, which operatively faces towards the palate of the person, includes a groove or bifurcation 128. A similar bifurcation (not shown) may be formed on the gripping or hooking contact surface 122. These bifurcations have been found to facilitate swallowing, whilst allowing passage of air, thereby further reducing the incidence of sleep apnoea and/or snoring. The bifurcations are formed in areas of the grip or hook member 120 which is operatively located at or near a median glossoepiglottic fold of the tongue 24 (especially during swallowing). The bifurcations typically do not extend linearly or along a sagittal plane. Instead, the bifurcations typically extend non-linearly and non-symmetrically relative to the grip or hook member 120. In portions of the grip or hook member 120, the bifurcations may be spaced from the sagittal plane. The shape of the bifurcation may be determined by taking an impression of the person, in use, when the person swallows.

In a variation which is not shown, the grip or hook member 120, when viewed from the top or bottom, extends slightly away from the sagittal plane, and therefore slightly oblique or at a tangent to the sagittal plane. Again, the grip or hook member 120 may extend non-symmetrically, which further facilitates swallowing.

A length of the grip or hook member 120 may be varied depending on anatomical features of the person.

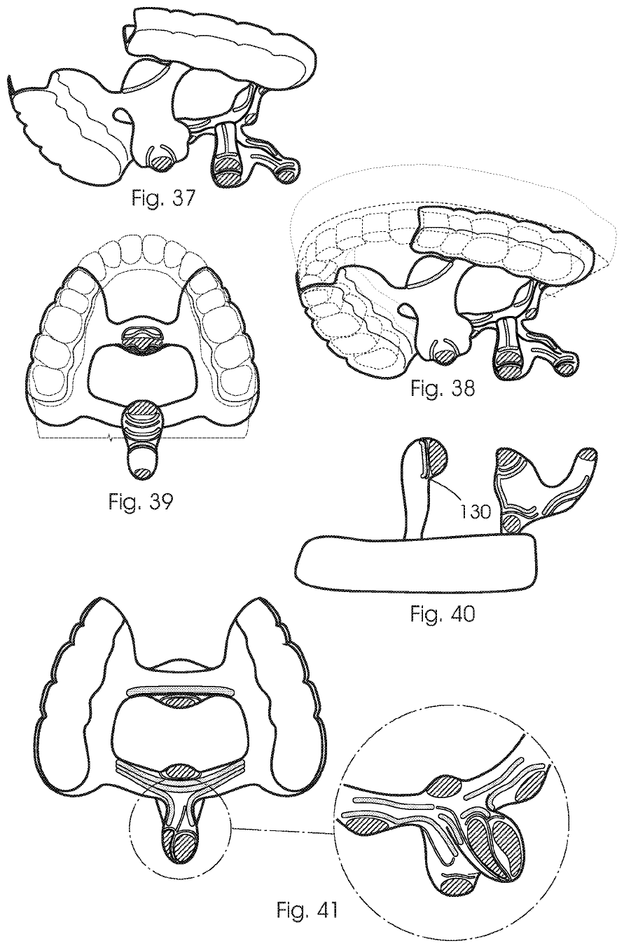

Surfaces of the device 10 may comprise various surface finishes and surface features. These finishes and features may serve to provide gripping or retention of the tongue relative to the device 10. FIGS. 37 to 41 show some example surface finishes and features on one of the example embodiments of the device 10. It will be appreciated that the specific shapes and configurations of these surface finishes and features are non-limiting, however, their presence may facilitate gripping and retention as discussed. Furthermore, even though only shown in respect of one of the embodiments illustrated herein, it will be appreciated that similar surface finishes and features may be present in any of the example embodiments of the invention. The surface finishes and features may generally be classified into: bulbosities or bulges (shown in FIGS. 37 to 41 by substantially oval patches with heavy hatching), ridges (shown in FIGS. 37 to 41 by elongate areas with light hatching), and hollows or grooves (shown in FIGS. 37 to 41 by elongate areas with dotted hatching).

In one example, the second protrusion 18.2 may comprise a hollow or hole formed on an operatively tongue-facing portion, at an area marked by reference numeral 130 in FIG. 40. The hollow or hole may be circular, partially circular or at least substantially circular, and may be provided for allowing a portion of the tongue to rest therein or protrude therethrough, to allow the tongue to be retained in position. The shape and configuration of the hollow may be determined based on the anatomy of the person and the shape of the tongue. The hollow may be relatively shallow, may extend about halfway, three quarters or completely through the second protrusion. The hollow provides an alternative retention configuration for retaining the tongue in a predetermined position. The tip of the tongue may therefore rest in or extend through the hollow, instead of extending all the way between the first and second protrusions. However, the first and second protrusions still both contact the tongue and cooperate to retain the tongue in the predetermined position.

It will be appreciated that the airway 68 may, absent the use of the device 10, become obstructed when the tongue moves to a rearward position and towards to the throat 26 of the person 16. It has been found that the use of the device 10 to retain the airway 68 substantially unobstructed, facilitates easier and more regulated breathing, which in turn, reduces the incidence or intensity of sleep-related breathing disorders, such as obstructive sleep apnoea. Furthermore, it has been found the use of the device 10 to retain the airway 68 substantially unobstructed, reduces the incidence and intensity, or even completely eliminates, snoring. This is achieved by the airway being opened by the interaction between the first protrusion and the tongue. The use of the device therefore inhibits contact between the tongue, the soft palate, and other tissues which normally vibrate during snoring. Also, the physical contact between the tongue and the first protrusion inhibits vibration of the rear parts of the tongue. Lastly, it has been found that the use of the device 10 could, in some cases, lessen the impact of a deviated septum, and could therefore simultaneously, in some cases, clear a nasal airway (not shown) which could, absent the use of the device 10, be obstructed due to a deviated septum.

It will be appreciated that the above description only provides a non-limiting number of example embodiments of the invention and that there may be many variations without departing from the spirit and/or the scope of the invention. For example, the embodiments in the figures are schematic representations only, as the specific appearance may vary depending on the anatomy of the person 16, and the like. Thicknesses, sizes, the presence of the various surface features and the like may all be adjusted according to individual anatomical features of the person. Furthermore, in some cases, the formation need not necessarily take the form of a protrusion as such, and the contact surface(s) may be formed directly on one or more formations extending from the retaining arrangement 12. In such cases, the formation may define a cross or bridge member, shaped to incorporate the relevant contact surface. It is believed that the device may furthermore serve as a tongue exercising device. The tongue may be used cyclically or repetitively to push the protrusion 18 forwards and pull same backwards against bias of the protrusion resulting from the flexibility of the material from which same is manufactured. This may aid in strengthening muscles of the tongue, which may again alleviate the incidence or intensity of sleep-related breathing disorders. Furthermore, it will be appreciated that an embodiment where the distal extension 50 is removed or shortened, may be feasible. The protrusion 18 may be solid or hollow. As shown in FIGS. 8 to 12, in some example embodiments, the cross-member 46 may comprise four connecting arms (indicated by reference numerals 46.1 to 46.4), which may serve to support the protrusion.

It will be appreciated that the device may urge or retain the tongue in alternative predetermined positions, which are not shown in the figures. Also, in some cases, the predetermined location 22 and contact surfaces may differ from those disclosed above. For example, it is believed that an embodiment where the tongue is wrapped around the protrusion, and where the tip of the tongue engages with and is retained by one of the connecting arms of the cross-member 46 (therefore towards the top of the mouth) may also be feasible. Alternative means of retaining the tip of the tongue relative to the device may therefore also be feasible.

Yet furthermore, reference to anatomical features and all dimensions provided in relation to anatomical features, are made and provided based on normal or standard human anatomy. The person 16, for the purpose of illustration of the technical details of the device 10, is therefore taken as a person without any anatomical abnormalities, deviations or deficiencies. It will, however, be appreciated that the device 10 may potentially find application despite certain anatomical abnormalities, deviations or deficiencies and the presence of such does not necessarily preclude use of the device 10. It will, furthermore, be appreciated that structural adaptations and adjustments may have to be made to the device, to render same functional in view of such anatomical abnormalities, deviations or deficiencies.

It will be easily understood from the present description that the particular features of the present invention, as generally described and illustrated in the figures, can be arranged and designed according to a wide variety of different configurations. In this way, the description of the present invention and the related figures are not provided to limit the scope of the invention but simply represent selected embodiments.

The skilled person will understand that the technical characteristics of a given embodiment can in fact be combined with characteristics of another embodiment, unless otherwise expressed or it is evident that these characteristics are incompatible. Also, the technical characteristics described in a given embodiment can be isolated from the other characteristics of this embodiment unless otherwise expressed.

1. A maxillary airway device, comprising:
a maxillary retaining arrangement for releasably retaining the device relative to a maxilla of a person;
at least a first formation extending from or supported by the retaining arrangement and comprising at least a first contact surface configured for operatively contacting at least a first predetermined location on a tongue of the person, thereby urging or retaining the tongue of the person towards or in a predetermined position.

2. The maxillary airway device according to claim 1, wherein the predetermined location on the tongue is located on an upper surface of a body of the tongue (corpus linguae).

3. The maxillary airway device according to claim 2, wherein the predetermined location is between a tip of the tongue and a region containing vallate papillae, and towards a medial portion of the corpus linguae.

4. The maxillary airway device according to claim 3, wherein the predetermined location is proximate the vallate papillae.

5. The maxillary airway device according to claim 1, wherein the predetermined location is vertically substantially in line with a junction between a hard palate and a soft palate of the person.

6. The maxillary airway device according to claim 1, wherein the predetermined location is vertically substantially in line with a lesser or greater palatine foramen of the person.

7. The maxillary airway device according to claim 1, wherein the predetermined location is proximal to a line connecting second molars of the person.

8. The maxillary airway device according to claim 1, wherein the predetermined location is between 30 mm and 60 mm from a tip of the tongue.

9. The maxillary airway device according to claim 1, wherein the predetermined location is proximate a location where central incisors contact the tongue when the tongue is extended as far forwards out of the mouth, as possible.

10. The maxillary airway device according to claim 1, wherein the predetermined location is between about 50 mm and 60 mm from a tip of the tongue.

11. The maxillary airway device according to claim 1, wherein the predetermined position is one of a neutral position of the tongue; a slightly forwards position, towards a bottom of the mouth, wherein the tip of the tongue contacts a lower incisor; and a deflected position.

12. The maxillary airway device according to claim 1, configured such that the first contact surface operatively exerts a downwards and/or forwards force on the tongue and wherein the first contact surface operatively deforms the tongue from a neutral position of the tongue.

13. The maxillary airway device according to claim 1, wherein operative interaction between the tongue and the at least first contact surface inhibits the tongue from lifting or moving towards a proximal or rear portion of the mouth, or towards a throat of the person.

14. The maxillary airway device according to claim 1, wherein the first formation includes a distal extension, which extends in a distal direction relative to the contact surface, and operatively, towards the tip of the tongue.

15. The maxillary airway device according to claim 14, wherein the distal extension has a slightly concave or cup-shaped surface.

16. The maxillary airway device according to claim 14, wherein the distal extension operatively extends at least partially along a length of the tongue to exert a distributed force on the tongue, thereby retaining same in the predetermined position.

17. The maxillary airway device according to claim 14, wherein the first formation includes a stem portion, and wherein the distal extension extends from the stem portion in cantilevered fashion.

18. The maxillary airway device according to claim 17, wherein a brace formation is located between the distal extension and the stem portion.

19. The maxillary airway device according to claim 14, wherein a level of a tongue-facing surface of the distal extension is spaced from a level of the contact surface by about 15 mm, wherein a tip or end of the distal extension extends about 30 mm distally from the contact surface and wherein the contact surface and/or distal extension have a width of about 10 mm.

20. The maxillary airway device according to claim 1, wherein a level of the contact surface is, in use, spaced from a palate of the person by between 50 mm and 60 mm.

21. The maxillary airway device according to claim 1, wherein the contact surface is, in use, spaced about 40 mm from maxillary incisors of the person.

22. The maxillary airway device according to claim 1, wherein a connecting line between the contact surface and incisors of the person is between 50 mm and 60 mm.

23. The maxillary airway device according to claim 1, wherein the retaining arrangement is configured to retain the device releasably relative to at least one of i) maxillary teeth of the person;
ii) an alveolar process; and iii) a maxillary dental arch in the form of maxillary gums or gingiva of the person.

24. The maxillary airway device according to claim 23, wherein the retaining arrangement comprises first and second retaining portions in the form of first and second retaining trays being substantially U-shaped in cross-section.

25. The maxillary airway device according to claim 24, wherein the first and second retaining portions are discrete portions.

26. The maxillary airway device according to claim 24, wherein the first and second portions are integrally formed to form a substantially U-shaped tray viewed from a bottom.

27. The maxillary airway device according to claim 24, wherein the retaining arrangement is one of: i) configured to retain the device releasably relative to first and second sets of maxillary teeth respectively, the first and second sets of maxillary teeth located on opposite sides of the mouth of the person; and ii) configured to retain the device releasably relative to first and second portions of a maxillary gingiva or gum of the person, the first and second portions of gingiva located on opposite sides of the mouth of the person.

28. The maxillary airway device according to claim 27, wherein the first and second sets of maxillary teeth comprise at least some of: i) first and second sets of maxillary molars and premolars; ii) maxillary canine teeth; and iii) maxillary third molars.

29. The maxillary airway device according to claim 28, wherein the first and second retaining portions extend beyond the second molars and/or the third molars at least partially over maxillary posterior tuberosities.

30. The maxillary airway device according to claim 1, being integrally formed from a medical grade and slightly elastic polymeric material.

31. The maxillary airway device according to claim 30, wherein the material is selected from a list comprising: i) a soft or substantially elastic material; ii) a dual laminate material; iii) printable resin; and iv) dental wire.

32. The maxillary airway device according to claim 30, wherein the material comprises one of: i) acrylic (polymethyl methacrylate); ii) nylon; iii) natural or synthetic rubber; iv) silicon; and v) a silicon-based material.

33. The maxillary airway device according to claim 1, further comprising a cross-member, bridge or brace, connecting opposite sides of the retaining arrangement or first and second retaining portions thereof.

34. The maxillary airway device according to claim 33, wherein a stem portion of the formation extends from the cross-member, bridge or brace.

35. The maxillary airway device according to claim 33, wherein the cross-member, bridge or brace comprises four arms.

36. The maxillary airway device according to claim 1, comprising a second formation which extends from or is supported by the retaining arrangement, the second formation comprising a second contact surface configured for operatively contacting a second predetermined location on a tongue of the person and wherein operative interaction between the first and second contact surfaces and the first and second predetermined locations respectively, urges or retains the tongue of the person towards or in the predetermined position.

37. The maxillary airway device according to claim 36, wherein the first and second formations are spaced apart and define a spacing distance therebetween.

38. The maxillary airway device according to claim 36, wherein the first and second formations are supported by first and second cross-members, bridges or bases respectively, which extend between opposite sides of the retaining arrangement or first and second retaining portions thereof.

39. The maxillary airway device according to claim 38, wherein the first and second cross-members, bridges or bases are spaced apart and define a spacing distance therebetween.

40. The maxillary airway device according to claim 37, wherein the second predetermined location is a location on an underside of the tongue and wherein the first and second contact surfaces operatively contact opposite sides of the tongue, such that at least a portion of the tongue is operatively received within the spacing distance.

41. The maxillary airway device according to claim 1, wherein the first formation comprises a first set made up of more than one protrusion and wherein the first contact surface is defined across the more than one protrusions of the first set.

42. The maxillary airway device according to claim 1, wherein the first formation includes an operatively throat-facing or rearward extending grip or hook member, defining a gripping or hooking contact surface, which is shaped and configured to contact a third predetermined location on the tongue which is towards a rear portion of the tongue or towards a throat of the person.

43. The maxillary airway device according to claim 42, wherein a palate-facing surface of the gripping or hook member includes a bifurcation.

44. The maxillary airway device according to claim 43, wherein the bifurcation extends non-linearly and at least in a portion thereof, spaced from an operative sagittal plane.

45. The maxillary airway device according to claim 1, including one or more portions with surface finishes selected from the list comprising: i) bulbosities; ii) bulges; iii) ridges; iv) bifurcations; v) hollows; and vi) grooves.

46. A method of manufacturing a device according to claim 1, the method comprising the steps of:
S1) determining a shape, profile, size and/or configuration of a structure associated with a maxilla of a person;
S2) determining a predetermined location on a tongue of the person;
S3) constructing the device, by:
S3.1) constructing a retaining arrangement based on the determined shape, profile, size and/or configuration of the structure; and
S3.2) constructing a formation based on the predetermined location.

47. The method according to claim 46, wherein step S1) comprises one of: i) taking an intraoral scan of a mouth of the person; and ii) taking a physical or dental impression of the mouth of the person.

48. The method of claim 46, wherein step S3 is conducted using an additive manufacturing process.

49. The method of claim 46, wherein step S3 comprises creating a mould of a portion of a mouth of the person and forming the retaining arrangement and formation on the mould.
